(19) United States
(12) Patent Application Publication
(10) Pub. No.: US 2025/0174300 A1
(43) Pub. Date: May 29, 2025

(54) System and Method for Predicting Protein Binding Using a Multi-Modal Prediction Model

(71) Applicant: Technology Innovation Institute – Sole Proprietorship LLC, Masdar City (AE)

(72) Inventors: Etienne Goffinet, Al Muneera (AE); Raghvendra Mall, Al Raha (AE); Ankita Singh, Al Raha (AE); Filippo Castiglione, Abu Dhabi (AE); Fabrizio Gabellieri, Al Raha (AE); Sen Yan, Abu Dhabi (AE); Haithem Boussaid, Abu Dhabi (AE)

(73) Assignee: Technology Innovation Institute – Sole Proprietorship LLC, Masdar City (AE)

(21) Appl. No.: 18/959,940

(22) Filed: Nov. 26, 2024

Related U.S. Application Data
(60) Provisional application No. 63/603,952, filed on Nov. 29, 2023.

(51) Int. Cl. G16B 15/30

(52) U.S. Cl. CPC G16B 15/30

(57) ABSTRACT

A method of predicting protein functions and interactions, including identifying a first protein and a second protein for analysis, generating a first embedded representation of a first amino acid sequence of the first protein and a second embedded representation of a second amino acid sequence of the second protein, generating a first contact map prediction for the first protein based on the first amino acid sequence and a second contact map prediction for the second protein based on the second amino acid sequence, generating first physiochemical features associated with the first protein, and second physiochemical features associated with the second protein, and generating a prediction score for a protein function or interaction between the first protein and the second protein based on the first embedded representation, the second embedded representation, the first contact map prediction, the second contact map prediction, the first physiochemical features and the second physiochemical features.

### CROSS-REFERENCE TO RELATED APPLICATIONS

This application claims priority to U.S. Provisional Application No. 63/603,952, filed Nov. 29, 2023, which is incorporated by reference in its entirety.

### FIELD OF DISCLOSURE

The present disclosure generally relates to a system and method for predicting protein binding using a multi-modal prediction model.

### BACKGROUND

Lymphocyte T-cells (either CD4 T-helpers or CD8 T cytotoxic) are key actors of the adaptive immunity of vertebrates. T-cell receptors (TCR) are cell membrane proteins that bind to fragments (called peptides) of an antigen that are presented by specialized antigen-presenting cells (APCs) such as macrophages and dendritic cells. In the initial phase of the immune response, the antigen (i.e., potentially pathogenic microorganism such as a virus or bacteria) is captured by APCs, processed, and attached to Major Histocompatibility Molecules (MHC). The resulting peptide-MHC complex (pMHC) is exposed on the cell surface to be presented to the T-cells. The interaction between the TCR and the pMHC that is required to activate the T-cells is highly specific and is a key step in the initiation of the immune response. The binding affinity can be effectively determined by two short amino acid (AA) sequences: a peptide fragment of eight or more residues of the antigen (hereinafter “antigen-epitope” or “epitope”); and its TCR counterpart. Within the TCR, the complementarity determining region 3 (CDR3) of TCRβ chain is known to be the component that binds with its cognate epitope pairs.

As proteins, both TCRs and epitopes process unique structural organizations, leading to the need for other representations or modalities in their representation. The primary structure of proteins refers to the linear arrangement of amino acid residues in the polypeptide chains. The secondary structure involves the folding of the polypeptide chain into regular structures such as alpha-helices and beta-strands, that are stabilized by hydrogen bonds between amino acid residues. The tertiary structure encompasses the three-dimensional conformation resulting from interactions between different regions and the polypeptide chain's second structural elements (helices and strands). For TCRs, the tertiary structure includes the arrangement of their variable and constant domains which is a key determinant in antigen recognition. Epitopes, on the other hand, fold into specific conformations dictated by the interactions between their amino acid residues. The tertiary structures of TCRs and epitopes are important for their proper functioning and interactions during immune responses, where TCRs recognize the antigen peptides presented by APCs.

### SUMMARY

In one aspect, the present disclosure relates to a method of predicting protein functions and interactions, comprising identifying, by a computing system, a first protein and a second protein for analysis, generating, by the computing system, a first embedded representation of a first amino acid sequence of the first protein and a second embedded representation of a second amino acid sequence of the second protein, generating, by the computing system, a first contact map prediction for the first protein based on the first amino acid sequence and a second contact map prediction for the second protein based on the second amino acid sequence, generating, by the computing system, first physiochemical features associated with the first protein, and second physiochemical features associated with the second protein, and generating, by the computing system, a prediction score for a protein function or interaction between the first protein and the second protein based on the first embedded representation, the second embedded representation, the first contact map prediction, the second contact map prediction, the first physiochemical features and the second physiochemical features.

In embodiments of this aspect, the disclosure according to any one of the above example embodiments, further comprising generating, by the computing system, the first contact map prediction for the first protein based on the first amino acid sequence and the second contact map prediction for the second protein based on the second amino acid sequence by estimating first three-dimensional distances between first amino acid residues in the first amino acid sequence, and estimating second three-dimensional distances between second amino acid residues in the second amino acid sequence.

In embodiments of this aspect, the disclosure according to any one of the above example embodiments, further comprising generating, by the computing system, the first physiochemical features associated with the first protein, and the second physiochemical features associated with the second protein by passing the first amino acid sequence and the second amino acid sequence through a set of descriptors.

In embodiments of this aspect, the disclosure according to any one of the above example embodiments, further comprising generating, by the computing system, the prediction score for the protein function or interaction between the first protein and the second protein by generating an embedded first protein amino acid sequence by encoding the first embedded representation of the first amino acid sequence, the first contact map prediction, and the first physiochemical features, and generating an embedded second protein amino acid sequence by encoding the second embedded representation of the second amino acid sequence, the second contact map prediction, and the second physiochemical features.

In embodiments of this aspect, the disclosure according to any one of the above example embodiments, further comprising concatenating the embedded first protein amino acid sequence and the embedded second protein amino acid sequence.

In embodiments of this aspect, the disclosure according to any one of the above example embodiments, further comprising generating, via a feed forward neural network, the prediction score based on the concatenated embedded first protein amino acid sequence and the embedded second protein amino acid sequence.

In embodiments of this aspect, the disclosure according to any one of the above example embodiments, further comprising receiving, by the computing system, input data comprising a protein sequence, a number of neighbors, and a classification threshold, initializing, by the computing system, a set of retrieved neighbors, for each modality of a plurality of modalities by extracting, by the computing system, a modality representation of the protein sequence, retrieving, by the computing system, neighbors using the modality representation and a corresponding vector database, and adding, by the computing system, the retrieved neighbors to the set of retrieved neighbors, computing, by the computing system, a probability for each label index based on the set of retrieved neighbors, and outputting, by the computing system, predicted labels having probabilities greater than or equal to the classification threshold.

In embodiments of this aspect, the disclosure according to any one of the above example embodiments, wherein the first protein is a T-cell receptor (TCR) and the second protein is an epitope.

In embodiments of this aspect, the disclosure according to any one of the above example embodiments, wherein the prediction score represents a binding affinity between the TCR and the epitope.

In embodiments of this aspect, the disclosure according to any one of the above example embodiments, wherein the first physiochemical features associated with the TCR and the second physiochemical features associated with the epitope include one or more of: hydrophobicity, charge, size, and polarity.

In embodiments of this aspect, the disclosure according to any one of the above example embodiments, wherein generating the first contact map prediction for the TCR and the second contact map prediction for the epitope comprises estimating spatial relationships between amino acid residues in the TCR and the epitope, respectively.

In one aspect, the present disclosure relates to a non-transitory computer-readable medium storing instructions that, when executed by one or more processors, cause the one or more processors to perform operations for predicting protein functions and interactions, the operations comprising identifying a first protein and a second protein for analysis, generating a first embedded representation of a first amino acid sequence of the first protein and a second embedded representation of a second amino acid sequence of the second protein, generating a first contact map prediction for the first protein based on the first amino acid sequence and a second contact map prediction for the second protein based on the second amino acid sequence, generating first physiochemical features associated with the first protein, and second physiochemical features associated with the second protein, and generating a prediction score for a protein function or interaction between the first protein and the second protein based on the first embedded representation, the second embedded representation, the first contact map prediction, the second contact map prediction, the first physiochemical features and the second physiochemical features.

In embodiments of this aspect, the disclosure according to any one of the above example embodiments, wherein the operations further comprise generating the first contact map prediction for the first protein based on the first amino acid sequence and the second contact map prediction for the second protein based on the second amino acid sequence by estimating first three-dimensional distances between first amino acid residues in the first amino acid sequence, and estimating second three-dimensional distances between second amino acid residues in the second amino acid sequence.

In embodiments of this aspect, the disclosure according to any one of the above example embodiments, wherein the operations further comprise generating the first physiochemical features associated with the first protein, and the second physiochemical features associated with the second protein by passing the first amino acid sequence and the second amino acid sequence through a set of descriptors.

In embodiments of this aspect, the disclosure according to any one of the above example embodiments, wherein the operations further comprise generating the prediction score for the protein function or interaction between the first protein and the second protein by generating an embedded first protein amino acid sequence by encoding the first embedded representation of the first amino acid sequence, the first contact map prediction, and the first physiochemical features, and generating an embedded second protein amino acid sequence by encoding the second embedded representation of the second amino acid sequence, the second contact map prediction, and the second physiochemical features.

In embodiments of this aspect, the disclosure according to any one of the above example embodiments, wherein the operations further comprise concatenating the embedded first protein amino acid sequence and the embedded second protein amino acid sequence.

In embodiments of this aspect, the disclosure according to any one of the above example embodiments, wherein the operations further comprise generating, via a feed forward neural network, the prediction score based on the concatenated embedded first protein amino acid sequence and the embedded second protein amino acid sequence.

In embodiments of this aspect, the disclosure according to any one of the above example embodiments, wherein the operations further comprise receiving input data comprising a protein sequence, a number of neighbors, and a classification threshold, initializing a set of retrieved neighbors, for each modality of a plurality of modalities extracting a modality representation of the protein sequence, retrieving neighbors using the modality representation and a corresponding vector database, and adding the retrieved neighbors to the set of retrieved neighbors, computing a probability for each label index based on the set of retrieved neighbors, and outputting predicted labels having probabilities greater than or equal to the classification threshold.

In embodiments of this aspect, the disclosure according to any one of the above example embodiments, wherein the first protein is a T-cell receptor (TCR) and the second protein is an epitope.

In embodiments of this aspect, the disclosure according to any one of the above example embodiments, wherein the prediction score represents a binding affinity between the TCR and the epitope.

In embodiments of this aspect, the disclosure according to any one of the above example embodiments, wherein the first physiochemical features associated with the TCR and the second physiochemical features associated with the epitope include one or more of: hydrophobicity, charge, size, and polarity.

In embodiments of this aspect, the disclosure according to any one of the above example embodiments, wherein generating the first contact map prediction for the TCR and the second contact map prediction for the epitope comprises estimating spatial relationships between amino acid residues in the TCR and the epitope, respectively.

In one aspect, the present disclosure relates to a system for predicting protein functions and interactions, comprising a processor, and a memory having programming instructions stored thereon, which, when executed by the processor, perform operations comprising identifying a first protein and a second protein for analysis, generating a first embedded representation of a first amino acid sequence of the first protein and a second embedded representation of a second amino acid sequence of the second protein, generating a first contact map prediction for the first protein based on the first amino acid sequence and a second contact map prediction for the second protein based on the second amino acid sequence, generating first physiochemical features associated with the first protein, and second physiochemical features associated with the second protein, and generating a prediction score for a protein function or interaction between the first protein and the second protein based on the first embedded representation, the second embedded representation, the first contact map prediction, the second contact map prediction, the first physiochemical features and the second physiochemical features.

In embodiments of this aspect, the disclosure according to any one of the above example embodiments, wherein the operations further comprise generating the first contact map prediction for the first protein based on the first amino acid sequence and the second contact map prediction for the second protein based on the second amino acid sequence by estimating first three-dimensional distances between first amino acid residues in the first amino acid sequence, and estimating second three-dimensional distances between second amino acid residues in the second amino acid sequence.

In embodiments of this aspect, the disclosure according to any one of the above example embodiments, wherein the operations further comprise generating the first physiochemical features associated with the first protein, and the second physiochemical features associated with the second protein by passing the first amino acid sequence and the second amino acid sequence through a set of descriptors.

In embodiments of this aspect, the disclosure according to any one of the above example embodiments, wherein the operations further comprise generating the prediction score for the protein function or interaction between the first protein and the second protein by generating an embedded first protein amino acid sequence by encoding the first embedded representation of the first amino acid sequence, the first contact map prediction, and the first physiochemical features, and generating an embedded second protein amino acid sequence by encoding the second embedded representation of the second amino acid sequence, the second contact map prediction, and the second physiochemical features.

In embodiments of this aspect, the disclosure according to any one of the above example embodiments, wherein the operations further comprise concatenating the embedded first protein amino acid sequence and the embedded second protein amino acid sequence, and generating, via a feed forward neural network, the prediction score based on the concatenated embedded first protein amino acid sequence and the embedded second protein amino acid sequence.

In embodiments of this aspect, the disclosure according to any one of the above example embodiments, wherein the operations further comprise receiving input data comprising a protein sequence, a number of neighbors, and a classification threshold, initializing a set of retrieved neighbors, for each modality of a plurality of modalities extracting a modality representation of the protein sequence, retrieving neighbors using the modality representation and a corresponding vector database, and adding the retrieved neighbors to the set of retrieved neighbors, computing a probability for each label index based on the set of retrieved neighbors, and outputting predicted labels having probabilities greater than or equal to the classification threshold.

In embodiments of this aspect, the disclosure according to any one of the above example embodiments, wherein the first protein is a T-cell receptor (TCR) and the second protein is an epitope.

In embodiments of this aspect, the disclosure according to any one of the above example embodiments, wherein the prediction score represents a binding affinity between the TCR and the epitope.

In embodiments of this aspect, the disclosure according to any one of the above example embodiments, wherein the first physiochemical features associated with the TCR and the second physiochemical features associated with the epitope include one or more of: hydrophobicity, charge, size, and polarity.

In embodiments of this aspect, the disclosure according to any one of the above example embodiments, wherein generating the first contact map prediction for the TCR and the second contact map prediction for the epitope comprises estimating spatial relationships between amino acid residues in the TCR and the epitope, respectively.

### BRIEF DESCRIPTION OF THE DRAWINGS

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1 is a block diagram illustrating an exemplary computing environment, according to example embodiments.

FIG. 2A is a block diagram illustrating an architecture of a prediction model, according to example embodiments.

FIG. 2B is a block diagram illustrating an encoder portion of a prediction model, according to example embodiments.

FIG. 3A is a flow diagram illustrating a method of multi-label classification for protein functions using retrieval-augmented classification, according to example embodiments.

FIG. 3B is a block diagram illustrating a classification pipeline, according to example embodiments.

FIG. 3C is a block diagram illustrating protein function training and prediction, according to example embodiments.

FIG. 4 is a block diagram illustrating a computing system, according to example embodiments.

FIG. 5 is a flow diagram illustrating a method of generating a binding affinity score between a T-cell receptor and an epitope, according to example embodiments.

FIG. 6A is a block diagram illustrating a computing device, according to example embodiments of the present disclosure.

FIG. 6B is a block diagram illustrating a computing device, according to example embodiments of the present disclosure.

FIG. 7A presents experimental results for an AntiMicrobial Peptide function prediction task, according to example embodiments of the present disclosure.

FIG. 7B presents more experimental results for an AntiMicrobial Peptide function prediction task, according to example embodiments of the present disclosure.

FIG. 7C presents more experimental results for an AntiMicrobial Peptide function prediction task, according to example embodiments of the present disclosure.

FIG. 7D presents more experimental results for an AntiMicrobial Peptide function prediction task, according to example embodiments of the present disclosure.

The features of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Accurate estimation of protein function and interactions plays an important role in many areas of biological research and therapeutic development. There have been several conventional machine learning approaches to predict various protein properties and interactions. For example, some conventional approaches rely on deep learning techniques with an evolution-based blocks substitution matrix (BLOSUM) to convert amino acids of protein sequences into numerical values. Other conventional approaches have employed pre-trained language models to summarize the embedding vectors on the amino acid level to obtain sequence-wise representations. Such approaches may be limited because they focus on one modality at a time (e.g., amino acid string, BLOSUM embedding, or physiochemical features).

One or more techniques disclosed herein may improve upon conventional machine learning approaches by performing a multi-modal attention-based prediction of protein functions and interactions. In such an approach, the textual representation of proteins, that are embedded with a pre-trained bi-directional encoder model, may be combined with two additional modalities: a comprehensive set of selected physiochemical properties and predicted contact maps that estimate the three-dimensional distances between amino acid residues in the sequences. This multi-modal approach may be applied to predict various protein properties and interactions, including but not limited to protein-protein binding, enzyme activity, solubility, stability, subcellular localization, and multiple functional annotations simultaneously. The system may utilize multi-modal vector databases for efficient storage and retrieval of protein-related information across different modalities. As an example application, the system can be used to predict TCR-epitope binding affinity for immunotherapy development.

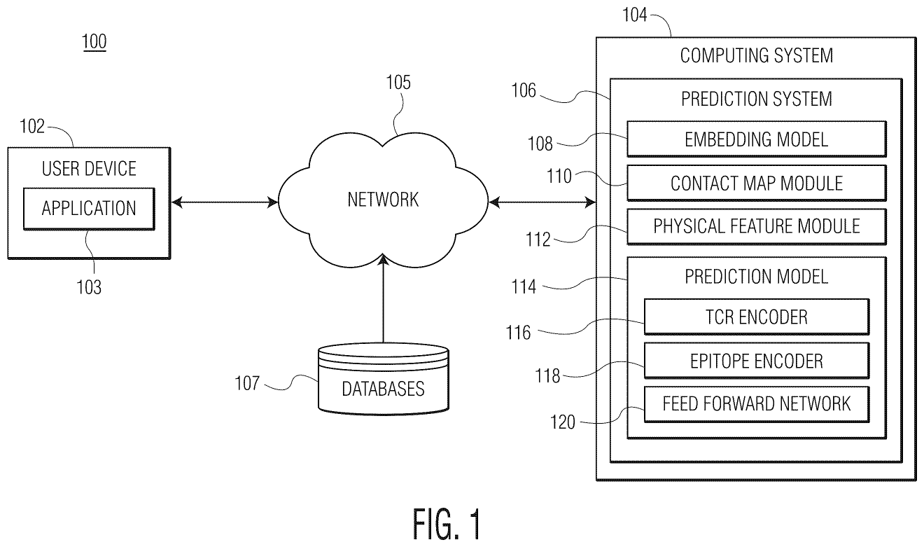

FIG. 1 is a block diagram illustrating a computing environment 100, according to example embodiments. As shown, computing environment 100 may include a user device 102 and a computing system 104 communicating via a network 105.

Network 105 may be representative of any suitable type, including individual connections via the Internet, such as cellular or Wi-Fi networks. In example embodiments, network 105 may connect terminals, services, and mobile devices using direct connections, such as radio frequency identification (RFID), near-field communication (NFC), Bluetooth™, low-energy Bluetooth™ (BLE), Wi-Fi™, ZigBee™, ambient backscatter communication (ABC) protocols, USB, WAN, or LAN. Because the information transmitted may be personal or confidential, security concerns may dictate one or more of these types of connection be encrypted or otherwise secured. In example embodiments, however, the information being transmitted may be less personal, and therefore, the network connections may be selected for convenience over security.

Network 105 may include any type of computer networking arrangement used to exchange data. For example, network 105 may be representative of the Internet, a private data network, virtual private network using a public network and/or other suitable connection(s) that enables components in computing environment 100 to send and receiving information between the components of computing environment 100.

User device 102 may be operated by a user. In example embodiments, user device 102 may represent devices of users that are associated with or subscribed to services offered by an entity associated with computing system 104. In example embodiments, user device 102 may be representative of one or more computing devices, such as, but not limited to, a mobile device, a tablet, a personal computer, a laptop, a desktop computer, or, more generally, any computing device or system having the capabilities described herein.

User device 102 may include application 103. Application 103 may be representative of a web browser that allows access to a website or a stand-alone application. User device 102 may access application 103 to access one or more functionalities of computing system 104. User device 102 may communicate over network 105 to request a webpage, for example, from a web client application server of computing system 104. In example embodiments, a user of user device 102 may upload information associated with a TCR and an epitope to computing system 104 to determine a binding affinity score between the TCR and the epitope.

Computing system 104 may be configured to host a prediction system 106. Prediction system 106 may be configured to predict TCR-epitope binding affinity. Prediction system 106 may be configured to treat both the TCR and the epitope as amino acid sequences. As such, prediction system 106 may apply the same pre-processing and multi-modal extraction process to both TCR and epitopes. The multi-modal extraction process may consider various modalities such as amino acid sequences, physiochemical features, and contact map.

As shown, prediction system 106 may include an embedding model 108, a contact map module 110, a physical feature module 112, and a prediction model 114. Each of contact map module 110 and physical feature module 112 may include of one or more software modules. The one or more software modules may be collections of code or instructions stored on a media (e.g., memory of computing system 104) that represent a series of machine instructions (e.g., program code) that implements one or more algorithmic steps. Such machine instructions may be the actual computer code the processor of computing system 104 interprets to implement the instructions or, alternatively, may be a higher level of coding of the instructions that may be interpreted to obtain the actual computer code. The one or more software modules may also include one or more hardware components. One or more aspects of an example algorithm may be performed by the hardware components (e.g., circuitry) itself, rather as a result of the instructions.

The system may also incorporate vector databases 107 for each modality to enable efficient storage and retrieval of protein-related information. In example implementations, the system may be capable of handling large, challenging datasets and may demonstrate improved scalability compared to conventional approaches. The multi-modal retrieval-augmented classification approach may significantly reduce prediction time, potentially enabling high-throughput analysis of protein functions.

Embedding model 108, contact map module 110, and physical feature module 112 may be configured to generate the inputs to prediction model 114 based on a first amino acid sequence representation of a protein or protein region and, in example cases, a second amino acid sequence representation of another protein or protein region. The system may be capable of processing single proteins for function prediction or protein pairs for interaction prediction.

Embedding model 108 may be configured to receive, as input, an amino acid sequence representation of both the TCR and the epitope and generate, as output, an equal length amino acid sequence representation of the TCR and epitope. For example, embedding model 108 may receive, as input, an amino acid sequence representation of the TCR, as a string, and an amino acid sequence representation of the epitope, as a string. The embedding model 108 may transform the amino acid sequence representations into a real-values vector. In example embodiments, the real-values vector may be of size 22×1024.

In example embodiments, embedding model 108 may be based on a bi-directional Long Short-Term Memory network (LSTM) that may leverage the amino acid context into the whole string. For example, embedding model 108 may be representative of the catELMo model. Because the attention block of prediction model 114 may be configured to receive equal-length amino acid sequences, embedding model 108 may be configured to truncate or pad each TCR and epitope sequence to a fixed context length (e.g., a length of 22).

Contact map module 110 may be configured to predict a contact map from an amino acid sequence representation of a given TCR and epitope. The predicted contact map representation may be used as a surrogate for the three-dimensional structure of a TCR/epitope complex. The motivation behind relying on contact maps may be two-fold: contact maps are lightweight (which helps control the computation cost) and invariant to rotation or translation. The contact map may be a matrix that may contain, in each cell (i,j), an estimate of the distance between the two residues (i,j). In other words, the predicted contact maps may estimate the three-dimensional distances between amino acid residues in the sequences. To generate the contact map prediction, contact map module 110 may utilize the evolution scale modeling package (ESM) on Python.

Physical feature module 112 may be configured to describe the amino acid sequence representations of TCR and epitope with a representation of physiochemical features through a set of selected descriptors. The physiochemical features may include physiochemical descriptors as well as global properties. Physical feature module 112 may extract the physiochemical features using the peptides package in Python.

In example embodiments, the physiochemical descriptors (e.g., nc=12) may include one or more of the following features (e.g., represented in a 75-dimensional space): BLOSUM indices, Cruciani properties, FASGAI vectors, Kidera factors, MS-WHIM scores, PCP descriptors, ProtFP descriptors, Sneath vectors, ST-scales, T-scales, VHSE-scales, and/or Z-scales. In example embodiments, these features may further include global properties (e.g., np=13) such as, but not limited to, one or more of aliphatic index, autocorrelation, autocovariance, Boman instability index, charge, hydrophobic moment α, hydrophobic moment β, hydrophobicity, instability index, isoelectric point, mass shift, molecular weight, and/or mass over charge ratio. In example embodiments, the obtained set of physiochemical and global properties may have a dimension of 88.

Prediction model 114 may be trained to predict the binding affinity between TCR and epitope based on the amino acid sequence embeddings, the predicted contact maps, and the physiochemical features. Prediction model 114 may be representative of an attention-based encoder that combines a self-attention mechanism of the encoder portion of a transformer architecture with a feed-forward neural network to generate a context-aware representation of its input. As shown, prediction model 114 may include a TCR encoder 116, an epitope encoder 118, and a feed-forward network 120.

TCR encoder 116 may be representative of an attention-based encoder. In example embodiments, the attention-based encoder may correspond to an encoder portion of a transformer architecture. TCR encoder 116 may be configured to receive, as input, the embedded TCR amino acid sequence, the predicted contact map for the TCR, and the physiochemical features of the TCR and generate, as output, an encoded representation of the TCR amino acid sequence−u.

Similarly, epitope encoder 118 may be representative of an attention-based encoder. In example embodiments, the attention-based encoder may correspond to an encoder portion of a transformer architecture. Epitope encoder 118 may be configured to receive, as input, the embedded epitope amino acid sequence, the predicted contact map for the epitope, and the physiochemical features of the epitope and generate, as output, an encoded representation of the epitope amino acid sequence−v.

The outputs of TCR encoder 116 and epitope encoder 118 may be concatenated together with their absolute difference |u−v|. The absolute difference between the encoded outputs may help focus prediction model 114 on the difference between the TCR and the epitope representation. The concatenated output may then be provided as input to feed-forward network 120. Feed-forward network 120 may output the binding affinity score between the TCR and the epitope.

While FIG. 1 was described with respect to TCR-epitope binding affinity prediction, the prediction system 106 may be expanded to perform multi-modal attention-based prediction of various protein functions and interactions. This expanded system may leverage the same underlying architecture and principles to analyze and predict a wide range of protein-related properties and interactions.

The expanded system may be capable of multi-label classification, allowing for the prediction of multiple protein functions simultaneously. This capability may enable the system to capture the complex, multi-functional nature of many proteins, providing a more comprehensive functional annotation. The system's performance may be evaluated using various metrics, including but not limited to Matthews Correlation Coefficient (MCC), sensitivity, specificity, balanced accuracy, and geometric mean, potentially demonstrating superior performance compared to existing baselines on challenging datasets.

In such an expanded system, the input to prediction system 106 may include amino acid sequences of any two proteins or protein regions of interest, not limited to TCRs and epitopes. The embedding model 108 may generate embedded representations of these input sequences, while the contact map module 110 and physical feature module 112 may extract relevant structural and physiochemical information, respectively.

The prediction model 114 may be adapted to handle different types of protein function and interaction predictions. For example, it may be trained on diverse datasets to predict protein-protein binding affinities, enzyme-substrate interactions, protein solubility, stability, or subcellular localization. The TCR encoder 116 and epitope encoder 118 may be generalized to become protein encoders, capable of processing any input protein sequence.

The feed-forward network 120 may be modified to output different types of predictions depending on the task at hand. For instance, it may generate a binding affinity score for protein-protein interaction predictions, a probability distribution over different cellular compartments for subcellular localization predictions, or a continuous value representing protein stability or solubility.

By maintaining the multi-modal approach that combines sequence information, predicted structural features, and physiochemical properties, this expanded system may capture a comprehensive representation of proteins. This may allow for more accurate and versatile predictions across various aspects of protein function and interaction, potentially aiding in areas such as drug discovery, protein engineering, and understanding cellular processes.

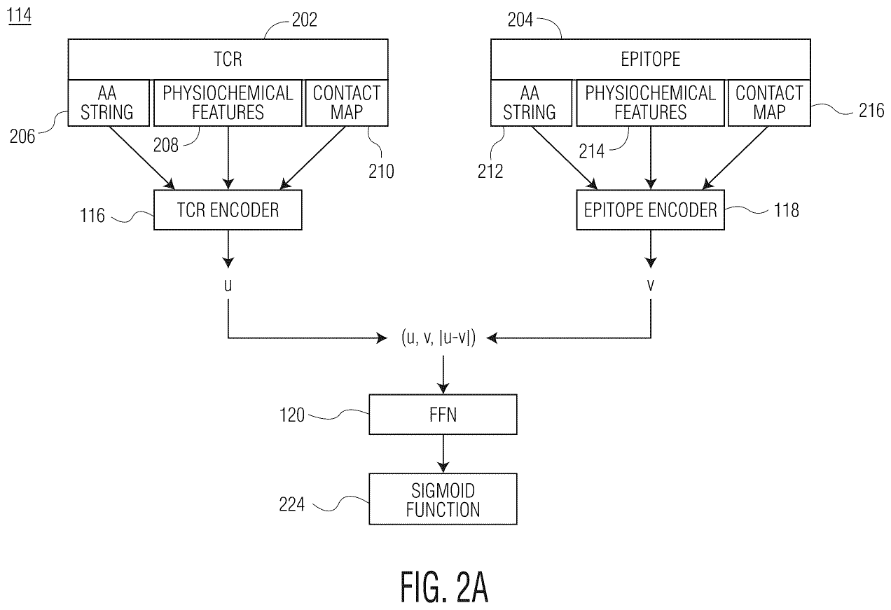

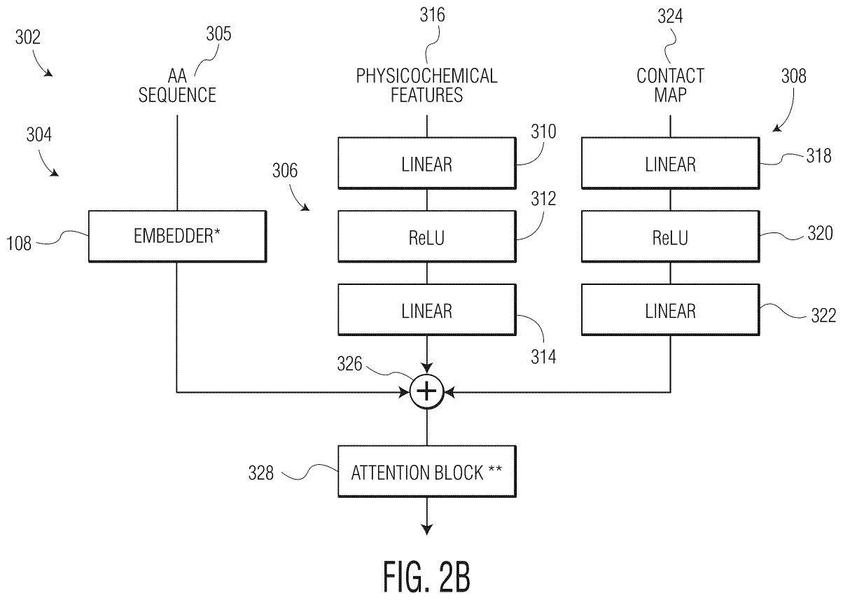

FIG. 2A is a block diagram illustrating the architecture of prediction model 114, according to example embodiments. Prediction model 114 may include TCR encoder 116, epitope encoder 118, and feed-forward network 120 (FFN). As shown, prediction model 114 may receive information associated with TCR 202 and information associated with epitope 204 as input. For TCR 202, amino acid string 206, physiochemical features 208, and contact map 210 may be provided, as input, to TCR encoder 116. TCR encoder 116 may generate, as output, an encoded representation u. Here, the output u may be the concatenation of three outputs: the output from embedding model 108, the output of physical feature portion 306, and the output of contact map portion 308. In example embodiments, rather than concatenating the outputs, the outputs may be summed, averaged, or added element-wise. Similarly, for epitope 204, amino acid string 212, physiochemical features 214, and contact map 216 may be provided, as input, to epitope encoder 118. Epitope encoder 118 may generate, as output, an encoded representation v. The encoded representation u may be concatenated with the encoded representation v and the absolute value of their difference, |u−v|, to generate the input (u, v, |u−v|).

The input (u, v, |u−v|) may be provided to feed-forward network 120. The output from feed-forward network 120 may pass through a sigmoid function 224. Prediction model 114 may then generate, as output, a binding affinity score for TCR 202 and epitope 204. In example embodiments, the binding affinity score may be between zero and one.

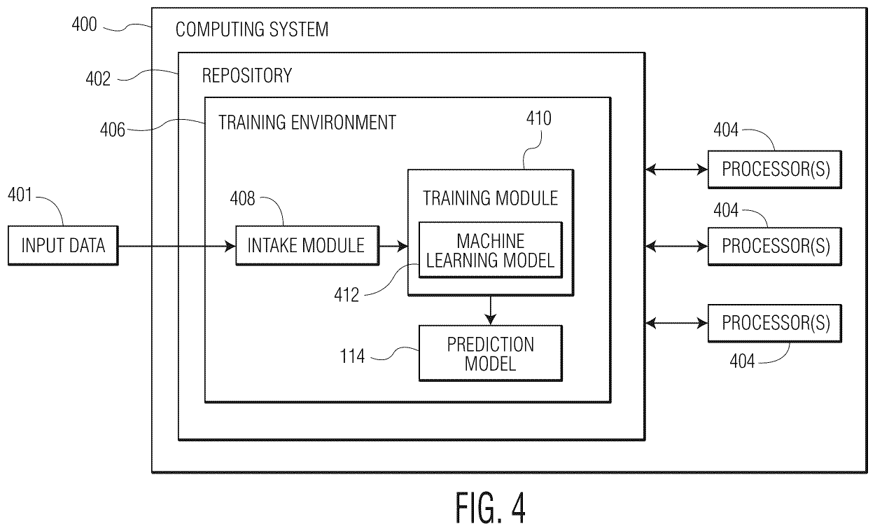

FIG. 2B is a block diagram illustrating an architecture of an encoder 302 of prediction model 114, according to example embodiments. Encoder 302 may represent the architecture utilized by both TCR encoder 116 and epitope encoder 118. As shown, encoder 302 may include embedding portion 304, physical feature portion 306, and contact map portion 308. Embedding portion 304 may include embedding model 108. As described above, embedding model 108 may be representative of a pre-trained bi-directional Long Short-Term memory network (LTSM) model, such as, but not limited to, the catELMo model. Embedding model 108 may be configured to receive, as input, an amino acid sequence string 305 and generate, as output, an equal length amino acid sequence representation of the TCR and epitope. Such embedding process may transform the amino acid sequence representations into a real-values vector. In example embodiments, such as that shown in FIG. 4, the real-values vector may be of size 22×1024.

Physical feature portion 306 may be representative of an encoder portion of a transformer network. As shown, physical feature portion 306 may include a first linear layer 310, a rectified linear unit (ReLU) activation function 312, and a second linear layer 314. Physical feature portion 306 may be configured to receive input generated by physical feature module 112. For example, physical feature portion 306 may receive, as input, physiochemical features 316. Physical feature portion 306 may generate, as output, physiochemical properties of the epitope and the TCR. The input to physical feature portion 306 may be a vector of physiochemical properties describing one amino-acid sequence (either the TCR or the epitope). The output of physical feature portion 306 may be a representation of this initial vector in a different space, produced by the linear layers and non-linear activation function. The output may be thought of as an encoding (or representation) of the initial physiochemical vector obtained by non-linear recombination.

Contact map portion 308 may be representative of an encoder portion of a transformer network. As shown, contact map portion 308 may include a first linear layer 318, a ReLU activation function 320, and a second linear layer 322. Contact map portion 308 may receive, as input, predicted contact map 324 generated by contact map module 110. Contact map portion 308 may generate, as output, a representation of predicted contact map 324 for the epitope and TCR. The input to contact map portion 308 may be a contact map that describes a TCR or an epitope. The output of contact map portion 308 may be a representation of the input in a different space.

Outputs from embedding portion 304, physical feature portion 306, and contact map portion 308 may be concatenated to generate a concatenated output 326. Such process may result in a 24×1024 tensor representation of the inputs, i.e., amino acid sequence string 305, physiochemical features 316, and predicted contact map 324. Concatenated output 326 may be provided, as input, to attention block 328.

Attention block 328 may be a beneficial component of prediction model 114. For example, given an input tensor X with dimension [symbol] ×d, the self-attention may be defined by:

$$Attn ( X ) = SoftMax \frac{Q . {K}^{T}}{\sqrt{{d}_{k}}} V$$

where the matrices Q, K, V share the same dimensions 1024×1024 and are linear projections of the same input matrix X. Such process may estimate the compatibility between the queries Q and the keys K and uses this compatibility to weight the values V. The Q, K and V matrices are projections obtained from the multiplication of a weight matrix with the same input X: Q=XW_{q}, K=XWₖ, V=XWᵥ. They may be interpreted as queries, keys and values matrices that aim to capture contextual information: in this case, how can one amino acid value be explained by its position and the content of the sequence it belongs to.

Accordingly, attention block 328 may be configured to output an attention aware representation of the amino acid sequences (i.e., one for the TCR and one for the epitope) based on concatenated output 326. Such attention aware representation of the amino acid sequences may be provided as input to feed-forward network 120, such as shown and described in conjunction with FIG. 2A.

The broader multi-modal solution for protein function prediction will now be described. This approach expands upon the TCR-epitope binding affinity prediction system to encompass a wider range of protein-related tasks. In this expanded system, a multi-modal representation of proteins may be utilized, incorporating three distinct modalities: protein sequence embedding, 3D structure representation, and physicochemical features. The protein sequence embedding may be generated using pre-trained protein language models, which may transform the amino acid sequence representations into real-valued vectors. These models may be trained on large corpora of protein sequence data and may learn to predict amino acids based on their context, thereby capturing meaningful representations of protein sequences.

The 3D structure of proteins may be represented using predicted contact maps. These contact maps may serve as a surrogate for the tertiary structure of a protein and may be both lightweight and invariant to rotation or translation. For a given amino acid sequence, the contact map may be a symmetric matrix containing estimates of the distances between residue pairs. This structural information may be generated using pre-trained language models capable of predicting contact maps from sequence data.

The physicochemical features of proteins may be described through a set of selected descriptors. These features may include both physicochemical descriptors and global properties of the protein. The physicochemical descriptors may encompass various indices and scales that capture different aspects of amino acid properties, while the global properties may include characteristics such as aliphatic index, hydrophobicity, and molecular weight.

To enable efficient retrieval and processing of this multi-modal protein representation, the system may utilize vector databases. Each modality—sequence embedding, contact map, and physicochemical features—may be stored in a dedicated vector database. These databases may be designed to efficiently store, manage, and index high-dimensional vector data, allowing for swift and low-latency queries.

The protein function prediction task may be framed as a multi-label classification problem, where multiple functions or properties may be assigned to each protein. This approach may allow for the prediction of complex functional profiles, as proteins often perform multiple roles simultaneously.

In aspects, the system may employ a retrieval-augmented classification approach. This method may involve querying pre-encoded protein information stored in vector databases to augment input data. The system may use a k-nearest neighbor (KNN) like process for classification, where label sets retrieved from multiple modalities are combined to generate label probabilities. This approach may improve scalability and reduce prediction time, enabling efficient processing of large-scale protein datasets.

The multi-modal approach described above provides a comprehensive framework for protein function prediction. The following paragraphs outline steps involved in this multi-modal approach, detailing how the system processes input data, retrieves relevant information, and generates predictions for protein functions. This approach integrates various data types and processing techniques to enhance the accuracy and reliability of protein function predictions.

The multi-modal solution may function as a Multi-Label Classifier that takes a single protein represented as a string of amino acid sequences as input and predicts its membership in one or more functional classes. This approach may leverage artificial intelligence models trained on a dataset of proteins with known functional labels, while also utilizing external databases of unlabeled proteins to enhance its predictive capabilities.

The system may process the input protein sequence through multiple modalities, including embedded representations, contact map predictions, and physiochemical features. These diverse representations may be combined and analyzed using advanced machine learning techniques, potentially including attention mechanisms and neural networks. The multi-modal approach may allow for a more comprehensive analysis of protein characteristics, enabling the system to predict a wide range of protein functions. Applications of this system may include predicting Biological Process, Cell Component, and Molecular Function categories as defined in benchmarks such as CAFA3, as well as specific functional properties like Antimicrobial Peptide activity. By integrating information from labeled training data and unlabeled protein databases, the system may achieve improved accuracy and generalization in protein function prediction tasks.

As mentioned above, the multi-modal solution may function as a Multi-Label Classifier that takes a single protein represented as a string of amino acid sequences as input and predicts its membership in one or more functional classes. This approach may leverage artificial intelligence models trained on a dataset of proteins with known functional labels, while also utilizing external databases of unlabeled proteins to enhance its predictive capabilities.

The system may process the input protein sequence through multiple modalities, including embedded representations, contact map predictions, and physiochemical features. These diverse representations may be combined and analyzed using advanced machine learning techniques, potentially including attention mechanisms and neural networks. The multi-modal approach may allow for a more comprehensive analysis of protein characteristics, enabling the system to predict a wide range of protein functions. Applications of this system may include predicting Biological Process, Cell Component, and Molecular Function categories as defined in benchmarks such as CAFA3, as well as specific functional properties like Antimicrobial Peptide activity. By integrating information from labeled training data and unlabeled protein databases, the system may achieve improved accuracy and generalization in protein function prediction tasks. Details of this multi-modal solution are now described in further detail.

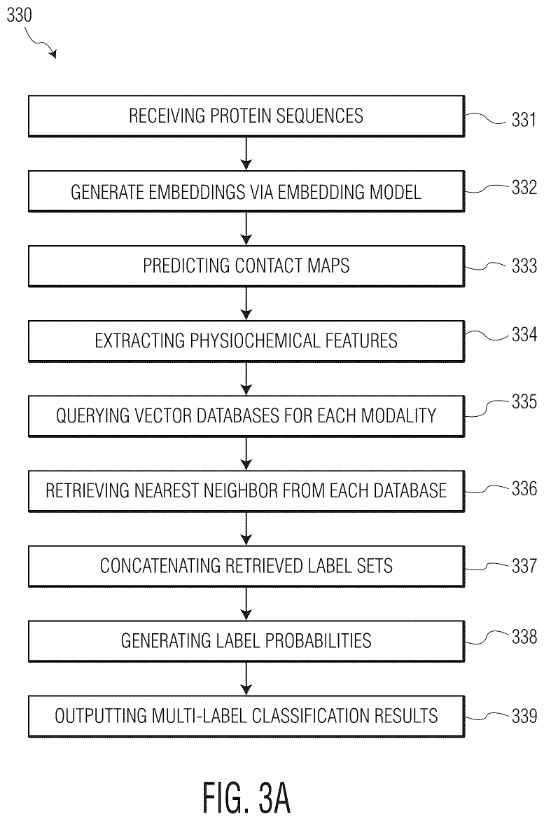

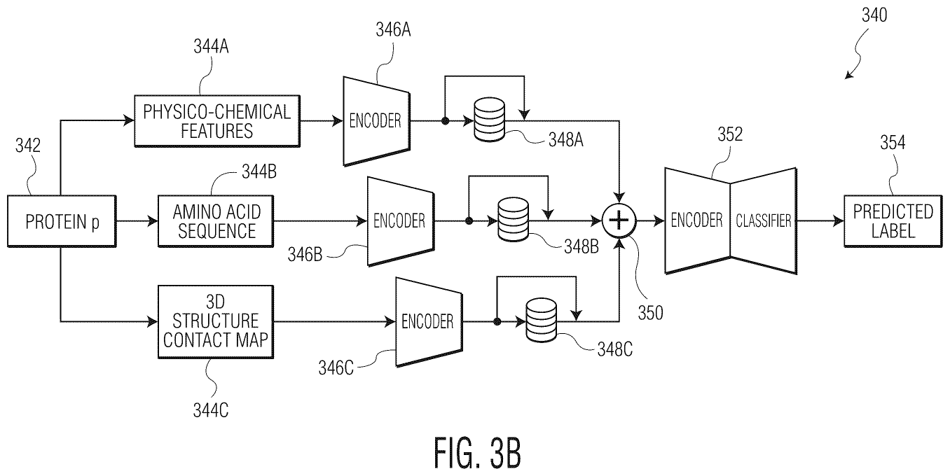

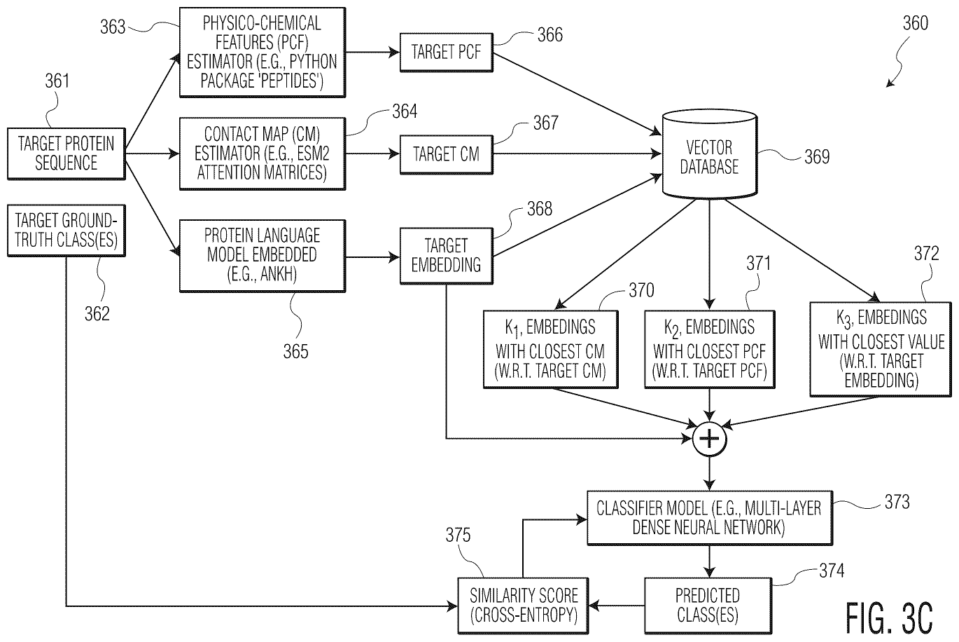

FIG. 3A illustrates a flowchart of a method 330 for multi-label classification of protein functions for building a database and using retrieval-augmented classification. The method may include several steps that process protein sequence data through multiple modalities to generate a multi-label classification result. Each step may be designed to extract and utilize different types of biological and chemical information from the protein sequences, contributing to a robust classification system.

The method 330 may generally include the steps of receiving protein sequences as input (step 331), generating embeddings for the input protein sequences (step 332), predicting contact maps for the protein sequences (step 333), extracting physiochemical features from the protein sequences (step 334), querying vector databases for each modality (step 335), retrieving the nearest neighbors from each database (step 336), concatenating the retrieved label sets from the different modalities (step 337), generating label probabilities based on the concatenated label sets (step 338), and outputting the multi-label classification results for the protein functions (step 339).

The method 330 may begin at step 331, where protein sequences are received as input. These sequences may represent the primary structure of proteins for which functional predictions are desired. At this step, the system may perform initial processing such as sequence validation, formatting standardization, and removal of any non-standard amino acid codes. The system may also handle different input formats.

In step 332, the method may generate embeddings for the input protein sequences using an embedding model. This embedding process may involve using pre-trained language models such as ProtBERT, ESM-1b, or UniRep. The embedding model may process the amino acid sequences through multiple transformer layers, capturing both local and global sequence contexts.

The resulting embeddings may be high-dimensional vectors that encode complex protein characteristics.

Step 333 may involve predicting contact maps for the protein sequences. This process may utilize deep learning models trained on protein structure databases. The contact map prediction may involve calculating the probability of spatial proximity between pairs (e.g. all pairs) of residues in the protein sequence. Advanced techniques like EigenTHREADER or trRosetta may be employed to generate these predictions. The resulting contact maps may be 2D matrices where each element represents the predicted distance or contact probability between two residues.

At step 334, the method may extract physiochemical features from the protein sequences. This step may involve calculating various numerical descriptors that capture properties such as hydrophobicity, charge, size, and polarity of amino acids. Tools like BioPython or the PROFEAT web server may be used to compute these features. The extracted features may include amino acid composition, dipeptide composition, pseudo-amino acid composition, and various physicochemical property groups. This step may result in a feature vector of large dimensions for each protein sequence.

Step 335 may involve querying vector databases for each modality. The system may use specialized vector databases optimized for high-dimensional data, such as FAISS or Annoy. For each modality (embeddings, contact maps, and physiochemical features), the system may perform nearest neighbor searches in the corresponding vector space. This step may involve techniques like approximate nearest neighbor search to efficiently handle large-scale datasets.

In step 336, the method may retrieve the nearest neighbors from each database. This retrieval process may use distance metrics appropriate for each modality, such as cosine similarity for embeddings or Frobenius norm for contact maps. The system may implement techniques like locality-sensitive hashing or product quantization to speed up the retrieval process. The number of neighbors retrieved may be a tunable parameter depending on the specific task and dataset size.

Step 337 may involve concatenating the retrieved label sets from the different modalities. This step may combine the functional annotations associated with the nearest neighbors found in each modality. The system may employ strategies to handle potential conflicts or redundancies in the retrieved labels, such as majority voting or weighted averaging based on the similarity scores of the retrieved neighbors.

At step 338, the method may generate label probabilities based on the concatenated label sets. This step may involve sophisticated aggregation techniques such as kernel density estimation or Gaussian mixture models to estimate the probability distribution over the label space. The system may also apply calibration techniques like Platt scaling or isotonic regression to ensure well-calibrated probability estimates.

In step 339, the method may output the multi-label classification results for the protein functions. This output may include a ranked list of predicted functions along with their associated probabilities. The system may apply thresholding techniques to determine which labels to assign, potentially using methods like F-measure optimization or precision-recall break-even point analysis. Additionally, the system may provide confidence intervals or other uncertainty estimates for each prediction to aid in interpretation and decision-making.

This multi-modal, retrieval-augmented approach may allow for a comprehensive analysis of protein characteristics, potentially leading to more accurate and diverse functional predictions. By leveraging information from pre-indexed databases across multiple modalities, the method may capture a wide range of protein properties that contribute to their functions.

While the flowchart in FIG. 3A provides a high-level overview of the multi-label classification process, it may be beneficial to examine a more detailed representation of the classification pipeline. FIG. 3B illustrates a specific implementation of the retrieval-augmented classification approach, showcasing how different modalities are processed and combined to generate protein function predictions.

The classification pipeline may involve a retrieval-augmented process, as illustrated in FIG. 3B. In this pipeline, a protein sequence 342 may be input to three parallel branches: a features block 344A, an embedding block 344B, and a contact map block 344C, and respective encoders 346A, 346B and 346C. Each of the respective encoders 346A, 346B and 346C may process the input to generate a vector representation, which may then be used to query the corresponding vector database such as feature vector database 348A, embedding vector database 348B, and contact map vector database 348C. The databases may return sets of labels associated with similar (e.g., most similar) stored vectors in the respective databases. These label sets may be concatenated at block 350, encoded into classifications at block 352 to produce a database of labels 354.

More specifically, the classification pipeline described in FIG. 3B represents an approach to protein function prediction that leverages multiple modalities of protein data. This retrieval-augmented process may begin with a single protein sequence input, which may be then processed through three distinct branches, each focusing on a different aspect of protein characterization.

The embedding encoder 346B may utilize protein language models to transform the amino acid sequence into a high-dimensional vector representation. This embedding may capture complex patterns and relationships within the protein sequence that may be relevant to its function. The prediction system may involve the creation of three distinct databases, each storing a dataset transformed to extract a specific modality of protein information. This multi-modal approach may allow for a more comprehensive representation of proteins, potentially leading to improved function prediction accuracy. This modality may be the protein primary representation, which is the sequence of amino acids encoded as a string. This string may be transformed into a matrix of float values, also known as embeddings, by feeding it through a Protein Language Model such as ESM-2. The embedding model may be a bidirectional transformer pre-trained on a large dataset of protein sequences. The pre-training process may involve inputting protein sequences with artificially masked sections and optimizing the reconstruction with respect to the ground truth.

The features encoder 346A may process the input to extract physiochemical properties of the protein. These features may include characteristics such as hydrophobicity, charge, size, and other relevant physicochemical attributes that can influence protein function and behavior. This modality may involve physicochemical properties of the proteins. These properties may be obtained through in-vivo or in-vitro sampling, or estimated using in-silico methods. In the case of in-silico estimation, computational methods from the literature may be used to approximate these properties.

The contact encoder 346C may generate a representation of the protein's predicted three-dimensional structure. This structural information may be beneficial for understanding potential interaction sites and functional domains within the protein. This modality may be the contact map, which represents the three-dimensional structure of the protein. For a given protein amino acid sequence, the contact map may be a matrix where each cell represents the Euclidean distance between pairs of amino acids in the 3D space. This contact map may be estimated in-vitro using structural biology methods such as X-ray crystallography, nuclear magnetic resonance, or cryo-electron microscopy. Alternatively, it may be estimated in-silico from the string representation. Recent research suggests that the attention matrices of a transformer model pre-trained on large protein datasets may approximate the contact map.

The encoder models may be pre-trained on a reference dataset, such as Uniref, using a reconstruction loss. Possible choices for the encoder may include auto-encoders and encoder transformers. In example cases, the encoder model may be fine-tuned on the multi-label classification training dataset. This fine-tuning process may help separate the embeddings and potentially improve the overall classification process. The system may utilize both the training dataset and a reference dataset of unlabelled proteins to enrich the database. The reference dataset may include public resources such as Uniref, ColabFoldDB, MgniFiy, BFD, or MetaClust, and may also be supplemented with private data. As a result of this approach, each amino acid sequence may be represented by a tuple consisting of a string, physical features, and a contact map. This multi-modal representation may provide a rich set of information about each protein, potentially enabling more accurate and comprehensive function predictions. In example implementations, additional modalities may be considered to further enhance the protein representation set. These may include multiple sequence alignments, which leverage protein homology, or alternative 3D structure representations such as graphs, geometries, and binding surfaces. The inclusion of these additional modalities may provide even more detailed information about protein structure and function, potentially improving the accuracy of predictions in certain contexts.

Each of these blocks may produce a vector representation of the protein from its respective perspective. These vectors may then be used to query corresponding vector databases (348A, 348B, 348C), which may be pre-populated with known proteins and their associated functional labels. The use of vector databases allows for efficient similarity searches in high-dimensional spaces, enabling the retrieval of proteins with similar characteristics across different modalities.

During a retrieval process, the databases (348A, 348B, 348C) may return sets of labels associated with similar (e.g., most similar) stored vectors. These labels may represent known functions or properties of proteins that share similarities with the input protein in terms of sequence, physicochemical features, or predicted structure. By retrieving labels from multiple modalities, the system may capture a more comprehensive view of potential protein functions.

The retrieval process for predicting labels associated with a target protein may involve several steps across multiple modalities. Initially, the target protein may undergo pre-processing to obtain embeddings for all possible modalities. For each modality, the system may compare the target protein's embedding to the embeddings stored in the corresponding database using a similarity score. This similarity score may be calculated using various metrics such as L1 norm, L2 norm, or cosine similarity, depending on the specific requirements of the task. The retrieval process may then proceed to select sets of elements. For example, it may retrieve the K closest embeddings from the database, where K is a user-specified parameter. This step may allow the system to identify proteins with similar characteristics across the chosen modality. Among the embeddings that have associated labels (typically those originating from the training dataset), the system may retrieve the K closest embeddings along with the frequency of occurrence for their associated labels. This approach may enable the system to leverage known functional annotations from similar proteins to inform the prediction for the target protein. In example implementations, the system may also retrieve embeddings based on a binding score with the target protein, potentially representing the ‘binding context’ of the target. This process may be repeated for each modality, and the retrieved lists of embeddings and label frequencies may be concatenated with the target protein's modality embeddings, creating a comprehensive representation for subsequent analysis and prediction.

The concatenation of these label sets at block 350 combines the information from the modalities (e.g., all three modalities). This step may allow the system to integrate evidence from different aspects of protein characterization, potentially leading to more robust predictions. The system may utilize a model that combines an encoder and a multi-label classifier network (see block 352) to generate predicted labels 354. This model may process the aggregated embeddings and labels obtained from the previous steps. The architecture of this model may incorporate various components such as Multi-Layer Perceptron classifiers, attention-based feature extraction layers, residual connections, and convolutional transformations. These components may work together to capture complex relationships within the data and generate accurate predictions across multiple labels simultaneously. The training process for this model may involve iterating over the training dataset and optimizing a Binary Cross-Entropy loss function. This loss function may be computed based on the label predictions generated by the model. Once calculated, the loss may be back-propagated through the layers of the encoder-classifier model using a neural network optimizer. The optimizer may be derived from the Stochastic Gradient Descent algorithm, with potential variants including Adam, Adagrad, or RMSProp. In example implementations, the backpropagation may stop at this stage, keeping the weights of the modality embedders frozen. However, the system may also include an option to use trainable instances of the modality embedders for extracting target protein modality representations, potentially allowing for fine-tuning of these components during the training process. The frequency of occurrence for each label across the retrieved sets may then be used to generate label probabilities 354. This approach effectively translates the multi-modal similarity search into a probabilistic prediction of protein functions. Labels that appear frequently across multiple modalities are likely to receive higher probabilities, reflecting a higher confidence in those functional predictions.

An example of a KNN classifier algorithm with multi-modal retrieval augmentation is described in Table 1 below where the vector database for each modality is input. This may include protein sequence, number of neighbors and classification threshold. The system may be then initialized to a set of retrieved neighbors. Each modality may be then operated on by repeatedly extracting modality representations of the protein sequence, retrieving neighbors using the modality and adding the retrieved neighbors to the set. Then, the method computes the probability for each label index and outputs the predicted label that is greater than or equal to the threshold.

TABLE 1
KNN classifier algorithm

```
Algorithm 1 KNN Classifier with Multi-Modal Retrieval Augmentation
  1: Input: Vector database for each modality M, [symbol]   ^{M} = {(Zᵢ^{M}, [symbol]   ᵢ)}, Test
    protein sequence Xₜₑₛₜ, Number of neighbors k, classification
    threshold t.
  2: Initialize: Set of retrieved neighbors [symbol]   = { }
  3: for each modality M in multi-modal sources do
  4:  Extract modality representation of Xₜₑₛₜ : [symbol]   ₜₑₛₜ^{M} =
        GetModality(Xₜₑₛₜ)
  5:  Retrieve neighbors using M: [symbol]   _{M} :: RetrieveNeighbors( [symbol]   ₜₑₛₜ^{M}, [symbol]
        ^{M}, k)
  6:  Add [symbol]   _{M} to [symbol]
  7: end for
  8; for each label index j do
  9:  P( [symbol]   _{i,j}) = 1) = 1/# [symbol]   Σ_{q∈} [symbol]   qj
10: end for
11: Output: Predicted label ypred : {P( [symbol]   _{i,j}) = 1) >= t}
```

Overall, this multi-modal, retrieval-augmented approach may allow for more comprehensive and accurate protein function predictions. By leveraging information from sequence, structure, and physicochemical properties, the system may capture a more complete representation of proteins. This may enable predictions across a wide range of protein-related tasks, potentially improving performance in areas such as enzyme function prediction, protein-protein interaction analysis, and protein engineering.

FIG. 3C illustrates a system diagram 360 for protein function training and prediction. The system may include several interconnected blocks that work together to process protein sequences and predict their functions. The blocks in the diagram may include: Target protein sequence 361, Target ground-truth classes 362, Physico-chemical features estimator 363, Contact map estimator 364, Protein language model embedder 365, Target PCF 366, Target CM 367, Target embedding 368, Vector database 369, Embeddings with closest CM 370, Embeddings with closest PCF 371, Embeddings with closest value 372, Classifier model 373, Predicted classes 374, and Similarity score 375. The system may operate in two modes: training and prediction.

In an example training mode, a target protein sequence 361 and its corresponding target ground-truth classes 362 may be provided as inputs. The target protein sequence 361 may be processed by three parallel modules: a physico-chemical features estimator 363 that generates target PCF 366, a contact map estimator 364 that produces target CM 367, and a protein language model embedder 365 that creates target embedding 368. The target PCF 366 may represent a set of physicochemical properties extracted from the protein sequence, while the target CM 367 may encode the predicted spatial relationships between amino acids in the protein structure. The target embedding 368 may capture contextual information about the protein sequence, potentially incorporating evolutionary and functional relationships learned from large-scale protein databases. These outputs may be used to query the vector database 369, which may retrieve three sets of embeddings: embeddings with closest CM 370, embeddings with closest PCF 371, and embeddings with closest value 372. The closest CM, PCF, and embeddings refer to the most similar representations found in the vector database for each modality. Specifically, embeddings with closest CM 370 are the database entries with contact maps most similar to the target protein's contact map, embeddings with closest PCF 371 are those with physicochemical features most similar to the target protein's features, and embeddings with closest value 372 are the database entries with sequence embeddings most similar to the target protein's embedding.

The retrieved embeddings, along with the target embedding 368, may be fed into the classifier model 373, which may output predicted classes 374. A similarity score 375 may be computed between the predicted classes 374 and the target ground-truth classes 362, which may be used to update and train the classifier model 373.

The training process may involve several iterations. In each iteration, the target protein sequence 361 may be processed through the three estimator modules (363, 364, 365) to generate the target representations (366, 367, 368). These representations may then be used to query the vector database 369, which may return the most similar embeddings based on each modality. The classifier model 373 may use these retrieved embeddings along with the target embedding to make predictions. The predicted classes 374 may be compared to the target ground-truth classes 362 using a similarity metric, generating a similarity score 375. This score may be used to update the parameters of the classifier model 373, potentially improving its performance over time. The process may be repeated for multiple target protein sequences, allowing the model to learn from a diverse set of examples. In example cases, the vector database 369 may be periodically updated with new embeddings to reflect the latest state of the model. This iterative training approach may allow the system to continuously refine its predictions and adapt to new data.

In the prediction mode, the system may receive a target protein sequence 361 and follow steps 363-372 to determine the closest CM, PCF and embeddings. The classifier model 373 may then output the predicted classes 374 for the input protein sequence. In this mode, blocks 362 and 375 may not be used, as there may be no ground-truth classes to compare against. This multi-modal approach may incorporate various aspects of protein characteristics (physico-chemical features, contact maps, and language model embeddings) to make comprehensive predictions about protein functions. The use of a vector database for retrieval may allow the system to leverage information from similar proteins, potentially improving prediction accuracy.

FIG. 4 is a block diagram illustrating a computing system 400, according to example embodiments. Computing system 400 may be representative of at least computing system 104 in FIG. 1. As shown, FIG. 4 may represent a training environment in which a prediction model may be trained to generate predictions for protein functions and interactions. In example aspects, this may include predicting binding affinity scores between proteins or protein regions, such as between TCRs and epitopes.

Computing system 400 may include a repository 402 and one or more computer processors 404. Repository 402 may be representative of any type of storage unit and/or device (e.g., a file system, database, collection of tables, or any other storage mechanism) for storing data. Further, repository 402 may include multiple different storage units and/or devices. The multiple different storage units and/or devices may or may not be of the same type or located at the same physical site. As shown, repository 402 includes a training environment 406. Training environment 406 may represent a computing environment in which the prediction model may be trained to generate predictions for protein functions and interactions, such as binding affinity scores between proteins or protein regions. Training environment 406 may include one or more of intake module 408 and training module 410.

Each of intake module 408 and training module 410 may include one or more software modules. The one or more software modules can be collections of code or instructions stored on a media (e.g., memory of computing system 400) that represent a series of machine instructions (e.g., program code) that implements one or more algorithmic steps. Such machine instructions may be the actual computer code the processor of computing system 400 interprets to implement the instructions or, alternatively, may be a higher level of coding of the instructions that are interpreted to obtain the actual computer code.

The one or more software modules may also include one or more hardware components. One or more aspects of an example algorithm may be performed by the hardware components (e.g., circuitry) itself, rather than as a result of the instructions. Intake module 408 may be configured to receive data for training. In example embodiments, the data used for training may be taken from databases of protein sequences, structures, and known interactions or functions. For example, the training data may include protein sequence pairs with interaction scores or functional annotations from one or more publicly available databases. In example embodiments, the training data set may include protein sequences, structural information, and functional annotations for various types of proteins and protein interactions. In example embodiments, to achieve a balanced dataset, intake module 408 may utilize protein sequences from repositories of known protein structures and functions. In example embodiments, intake module 408 may generate the training data set by combining data from various protein databases and resources. In example embodiments, intake module 408 may generate the training data set by providing the data from the various databases to one or more of an embedding model, a contact map module, and a physical feature module. In this manner, intake module 408 may generate a robust training set for each protein or protein pair that includes the amino acid sequence, an embedded representation of the amino acid sequence, a contact map prediction, physiochemical features, and known functional annotations or interaction scores. Such data may form the training data set for training the prediction model.

Training module 410 may be configured to train a machine learning model 412 to generate predictions for protein functions and interactions based on the training data generated by intake module 408. In example embodiments, the training process may be a supervised training process. For example, training module 410 may provide labels in the form of known functional annotations or interaction scores.

Once trained, the prediction model 114 may be ready for deployment in a computing system. For example, the prediction model 114 may be loaded onto a field programmable gate array (FGPA), a graphics processing unit (GPU), an application-specific integrated circuit (ASIC), and the like.

When expanding to the broader solution of a multi-modal attention-based prediction of protein functions and interactions, the blocks in FIG. 4 may be modified in several ways:

Intake module 408 may be expanded to handle a wider range of protein-related data. Instead of focusing solely on TCR-epitope pairs, it may process diverse protein sequences, structures, and functional annotations from various databases. The module may incorporate data from sources such as UniProt, PDB, Gene Ontology, and other specialized databases relevant to specific protein functions and interactions.

The training data set generated by intake module 408 may include a broader range of features for each protein. In addition to amino acid sequences, embedded representations, contact map predictions, and physiochemical features, it may also include information on protein domains, evolutionary conservation, post-translational modifications, and interaction partners.

Embedding model 108 may be adapted to generate more comprehensive protein sequence embeddings. It may utilize advanced pre-trained language models specifically designed for protein sequences, capturing complex patterns and relationships within the amino acid sequences.

Contact map module 110 may be enhanced to predict more detailed structural information. In addition to contact maps, it may generate predictions for secondary structure elements, solvent accessibility, and disorder regions. These structural features may provide a more comprehensive representation of the protein's 3D structure.

Physical feature module 112 may be expanded to include a wider range of physiochemical properties and global features relevant to various protein functions and interactions. This may include properties related to protein stability, binding affinity, and enzymatic activity.

Training module 410 may be modified to support multi-task learning, allowing the model to simultaneously predict multiple protein functions and interaction properties. This may involve adapting the loss function and training process to handle multiple output labels or continuous values.

Machine learning model 412 may be redesigned as a more complex architecture capable of handling the increased complexity and diversity of the multi-modal input data. This may include incorporating additional attention mechanisms, graph neural networks for processing structural information, or transformer-based architectures for capturing long-range dependencies in protein sequences.

Repository 402 may be expanded to include dedicated vector databases for efficient storage and retrieval of the multi-modal protein representations. This may involve implementing specialized indexing and search algorithms optimized for high-dimensional protein feature vectors.

The system may also include additional modules for post-processing and interpreting the model's predictions. These modules may integrate predictions across multiple modalities, provide confidence scores, and generate explanations or visualizations to aid in the interpretation of the predicted protein functions and interactions.

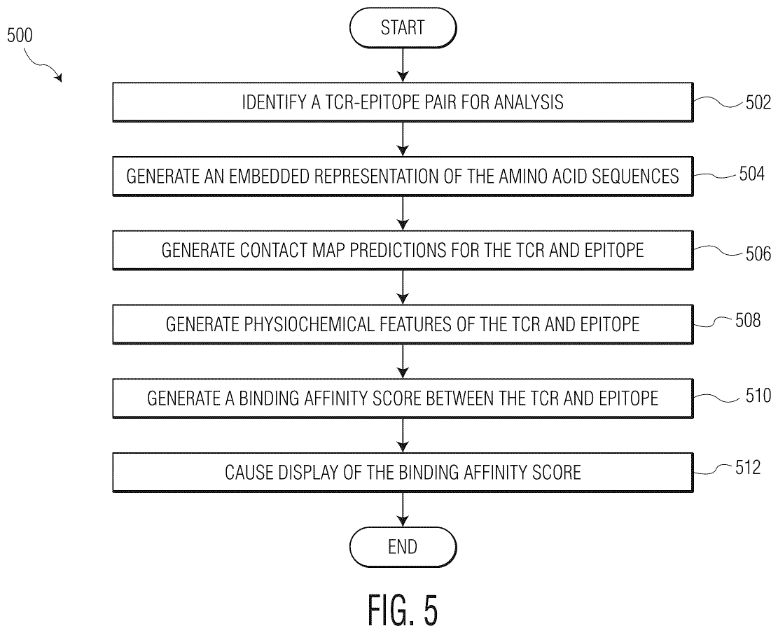

FIG. 5 is a flow diagram illustrating a method 500 of generating a binding affinity score between a TCR and an epitope, according to example embodiments. Method 500 may begin at step 502.

At step 502, computing system 104 may identify a TCR-epitope pair for analysis. In example embodiments, computing system 104 may identify a TCR-epitope pair for analysis via a user operating computing system 104. In example embodiments, computing system 104 may identify a TCR-epitope pair for analysis based on a request from an external client device. For example, user device 102 may request that a TCR-epitope pair be analyzed by prediction system 106 for determining a binding affinity score between the TCR and epitope. In example embodiments, the TCR-epitope pair may include an amino acid sequence of the TCR and an amino acid sequence of the epitope.

At step 504, computing system 104 may generate an embedded representation of the amino acid sequence of the TCR and the amino acid sequence of the epitope. Embedding model 108 may receive, as input, an amino acid sequence representation of the TCR and generate, as output, an equal length amino acid representation of the TCR. Embedding model 108 may also receive, as input, an amino acid sequence of the epitope and generate, as output, an equal length amino acid sequence of the epitope. Such embedding process may transform the amino acid sequence representations into a real-values vector.

At step 506, computing system 104 may generate contact map predictions for the TCR and the epitope. The generated contact map representations may be used as a surrogate for the three-dimensional structure of a TCR/epitope complex. To generate the contact map predictions, contact map module 110 may utilize the ESM package on Python.

At step 508, computing system 104 may generate physiochemical features of the TCR and the epitope. Physical feature module 112 may generation a description of the amino acid sequence representations of the TCR and the epitope with a representation of physiochemical features through a set of selected descriptors. The physiochemical features may include physiochemical descriptors as well as global properties. Physical feature module 112 may extract the physiochemical features using the peptides package in Python.

At step 510, computing system 104 may generate a binding affinity score between the TCR and the epitope based on the embedded representation of the amino acid sequence, the contact map prediction, and the physiochemical features of the TCR and the epitope. In example embodiments, TCR encoder 116 generate an encoded representation of the TCR amino sequence (u) based on the embedded TCR amino acid sequence, the predicted contact map for the TCR, and the physiochemical features of the TCR and generate, as output, an encoded representation of the TCR amino acid sequence−u. Epitope encoder 118 may generate an encoded representation of the epitope amino acid sequence (v), based on the embedded epitope amino acid sequence, the predicted contact map for the epitope, and the physiochemical features of the epitope. The outputs of TCR encoder 116 (e.g., u) and epitope encoder 118 (e.g., v) may be concatenated together with their absolute difference |u−v|. Based on the concatenated output, feed-forward network 120 may generate the binding affinity score between the TCR and the epitope.

At step 512, computing system 104 may cause display of the binding affinity score between the TCR and the epitope. For example, computing system 104 may cause display of the binding affinity score on a display associated with computing system 104 or client device 102.

When expanding to the broader solution of a multi-modal attention-based prediction of protein functions and interactions, the steps in FIG. 5 may be modified as follows:

At step 502, instead of identifying a TCR-epitope pair, the computing system may identify a protein or a pair of proteins for analysis. This may include various types of proteins or protein complexes, not limited to TCR-epitope pairs.

At step 504, the computing system may generate embedded representations for a wider range of protein sequences. The embedding model may utilize more advanced pre-trained language models specifically designed for diverse protein sequences, capturing complex patterns and relationships within the amino acid sequences.

At step 506, the contact map predictions may be expanded to include more detailed structural information. In addition to contact maps, the system may generate predictions for secondary structure elements, solvent accessibility, and disorder regions. These structural features may provide a more comprehensive representation of the protein's 3D structure.

At step 508, the generation of physiochemical features may be expanded to include a wider range of properties relevant to various protein functions and interactions. This may include features related to protein stability, binding affinity, enzymatic activity, and other relevant characteristics.

At step 510, instead of generating a binding affinity score, the system may predict multiple protein functions and interaction properties simultaneously. This may involve using a more complex prediction model capable of handling multi-modal input data and producing multiple output predictions. The model may incorporate additional attention mechanisms, graph neural networks for processing structural information, or transformer-based architectures for capturing long-range dependencies in protein sequences.

At step 512, the system may display a range of predicted protein functions and interaction properties, rather than just a binding affinity score. This may include confidence scores for each prediction, as well as visualizations or explanations to aid in the interpretation of the results.

Additionally, the method may include new steps such as: Retrieving relevant information from expanded databases and vector stores that contain diverse protein-related data, Integrating predictions across multiple modalities to provide a comprehensive functional profile of the analyzed proteins, and Applying post-processing techniques to refine predictions and generate interpretable results for various protein functions and interactions.

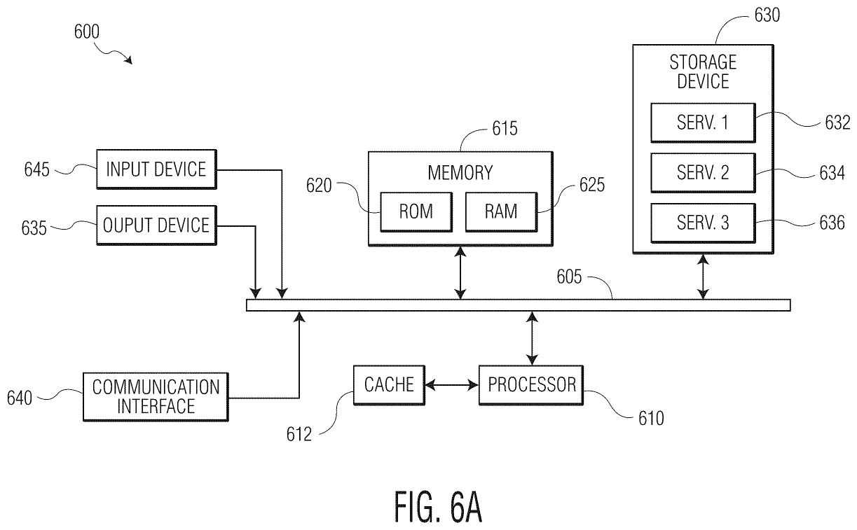

FIG. 6A illustrates a system bus architecture of computing system 600, according to example embodiments. System 600 may be representative of at least computing system 104. One or more components of system 600 may be in electrical communication with each other using a bus 605. System 600 may include a processing unit (CPU or processor) 610 and a system bus 605 that couples various system components including the system memory 615, such as read only memory (ROM) 620 and random-access memory (RAM) 625, to processor 610.

System 600 may include a cache of high-speed memory connected directly with, in close proximity to, or integrated as part of processor 610. System 600 may copy data from memory 615 and/or storage device 630 to cache 612 for quick access by processor 610. In this way, cache 612 may provide a performance boost that avoids processor 610 delays while waiting for data. These and other modules may control or be configured to control processor 610 to perform various actions. Other system memory 615 may be available for use as well. Memory 615 may include multiple different types of memory with different performance characteristics. Processor 610 may include any general-purpose processor and a hardware module or software module, such as service 1 632, service 2 634, and service 3 636 stored in storage device 630, configured to control processor 610 as well as a special-purpose processor where software instructions are incorporated into the actual processor design. Processor 610 may essentially be a completely self-contained computing system, containing multiple cores or processors, a bus, memory controller, cache, etc. A multi-core processor may be symmetric or asymmetric.

To enable user interaction with the computing system 600, an input device 645 may represent any number of input mechanisms, such as a microphone for speech, a touch-sensitive screen for gesture or graphical input, keyboard, mouse, motion input, speech and so forth. An output device 635 may also be one or more of a number of output mechanisms known to those of skill in the art. In instances, multimodal systems may enable a user to provide multiple types of input to communicate with computing system 600. Communications interface 640 may generally govern and manage the user input and system output. There is no restriction on operating on any particular hardware arrangement and therefore the basic features here may easily be substituted for improved hardware or firmware arrangements as they are developed.

Storage device 630 may be a non-volatile memory and may be a hard disk or other types of computer readable media which may store data that are accessible by a computer, such as magnetic cassettes, flash memory cards, solid state memory devices, digital versatile disks, cartridges, random access memories (RAMs) 625, read only memory (ROM) 620, and hybrids thereof.

Storage device 630 may include services 632, 634, and 636 for controlling the processor 610. Other hardware or software modules are contemplated. Storage device 630 may be connected to system bus 605. In one aspect, a hardware module that performs a particular function may include the software component stored in a computer-readable medium in connection with the necessary hardware components, such as processor 610, bus 605, output device 635 (e.g., display), and so forth, to carry out the function.

FIG. 6B illustrates a computer system 650 having a chipset architecture that may represent user device 102. Computer system 650 may be an example of computer hardware, software, and firmware that may be used to implement the disclosed technology. System 650 may include a processor 655, representative of any number of physically and/or logically distinct resources capable of executing software, firmware, and hardware configured to perform identified computations. Processor 655 may communicate with a chipset 660 that may control input to and output from processor 655.

In this example, chipset 660 outputs information to output 665, such as a display, and may read and write information to storage device 670, which may include magnetic media, and solid-state media, for example. Chipset 660 may also read data from and write data to storage device 675 (e.g., RAM). A bridge 680 for interfacing with a variety of user interface components 685 may be provided for interfacing with chipset 660. Such user interface components 685 may include a keyboard, a microphone, touch detection and processing circuitry, a pointing device, such as a mouse, and so on. In general, inputs to system 650 may come from any of a variety of sources, machine generated and/or human generated.

Chipset 660 may also interface with one or more communication interfaces 690 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 655 analyzing data stored in storage device 670 or storage device 675. Further, the machine may receive inputs from a user through user interface components 685 and execute appropriate functions, such as browsing functions by interpreting these inputs using processor 655.

It may be appreciated that example systems 600 and 650 may have more than one processor 610 or be part of a group or cluster of computing devices networked together to provide greater processing capability.

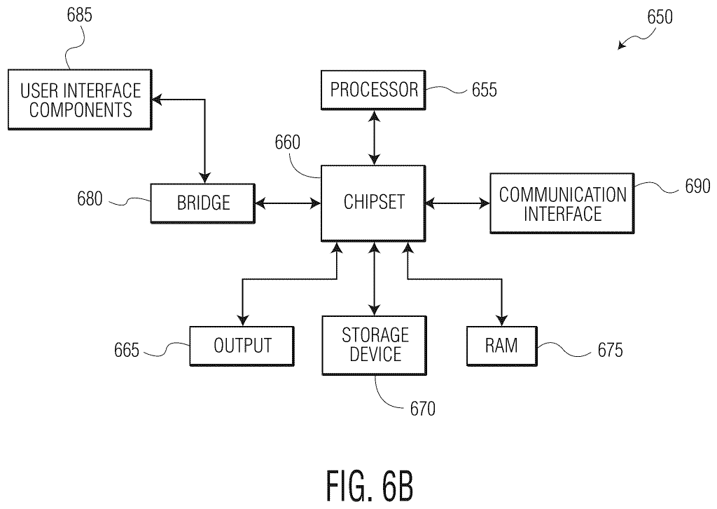

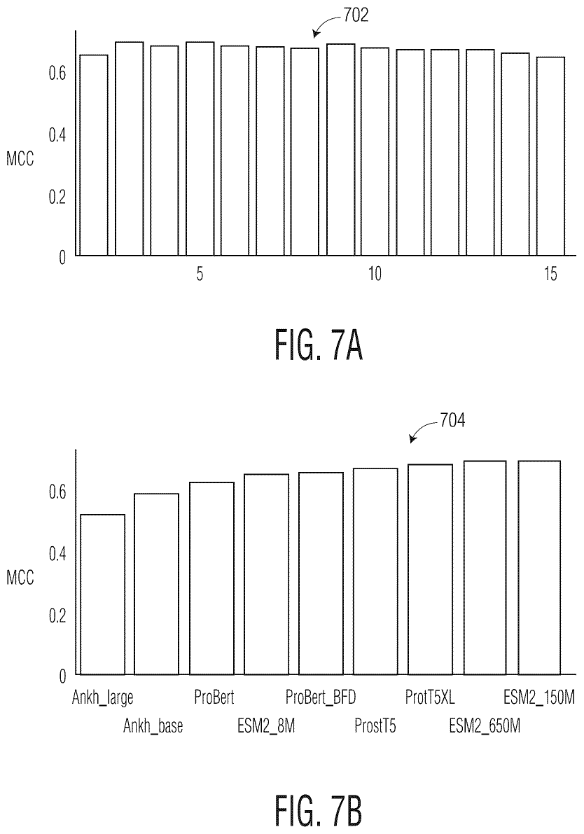

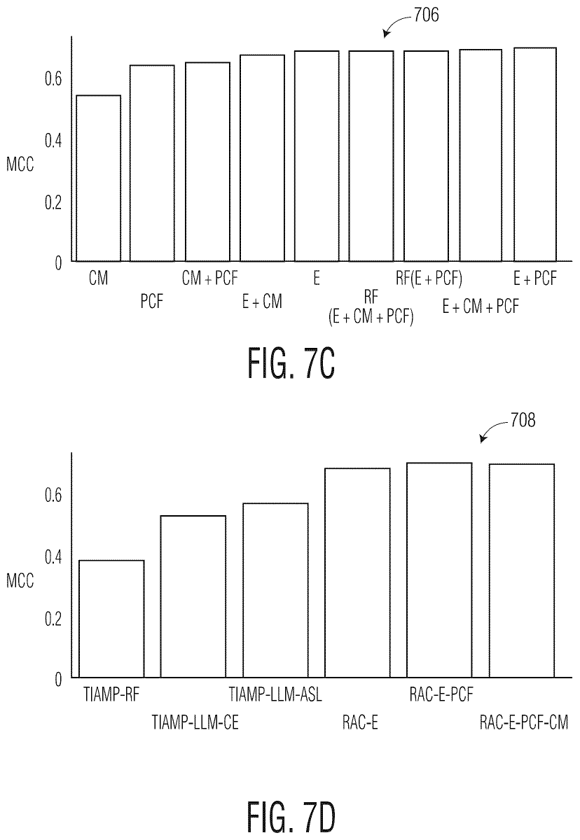

FIGS. 7A-7D presents experimental results 702, 704, 706, 708 for the AntiMicrobial Peptide function prediction task.

For ease of reference, the acronyms used in FIGS. 7A-7D may be defined as follows: Ankh_large and Ankh_base: Variants of the Ankh protein language model, with “large” and “base” referring to different model sizes. ProtBert: A protein language model based on the BERT (Bidirectional Encoder Representations from Transformers) architecture. ESM2_8M, ESM2_650M, ESM2_150M: Variants of the Evolutionary Scale Modeling (ESM) protein language model, with the numbers indicating the approximate number of parameters (e.g., 8 million, 650 million, 150 million). ProtBert_BFD: A variant of ProtBert trained on the BFD (Big Fantastic Database) dataset. ProtT5, ProtT5XL: Protein language models based on the T5 (Text-to-Text Transfer Transformer) architecture, with XL indicating an extra-large version. CM: Contact Map, a representation of protein structure. PCF: Physicochemical Features, describing various chemical and physical properties of proteins. E: Embedding, referring to the vector representation of protein sequences. RF: Random Forest, a machine learning algorithm. TIAMP: A baseline model for antimicrobial peptide prediction. LLM-ASL: A Large Language Model with Adaptive Synthetic Learning. RAC: Retrieval-Augmented Classification, the proposed approach in this study. The combinations (e.g., E+CM, E+PCF) indicate different combinations of input modalities used in the experiments.

In general, bar chart 702 shows the impact of the number of retrieved examples on the model's performance, measured by the MCC metric. This panel may provide insights into how the model's accuracy changes as more examples are used for prediction. Bar chart 704 compares the performance of different protein sequence embedders, also using the MCC metric. This comparison may help identify which embedding techniques are most effective for this particular task. Bar chart 706 demonstrates the effects of different modality variants on the model's performance, again using MCC. This panel may illustrate how combining different types of input data affects the model's predictive capabilities. Bar chart 708 compares the best model from the experiments with previous baselines, showing a significant performance improvement. This comparison may highlight the advancements made by the new approach over existing methods.

It is noted that the experimentation dataset used in generating FIGS. 7A-7D includes a collection of small proteins exhibiting various anti-microbial functions, including anticancer, antifungal, anti-gram-negative bacterial, anti-gram-positive bacterial, anti-mammalian, anti-parasitic, and antiviral properties. This dataset, totaling 6,460 proteins, was compiled from several anti-microbial peptide databases. The dataset exhibits significant imbalance, with the least frequent label (anti-parasitic) occurring in only 2.85% of cases, while the most frequent labels (anti-gram-negative and anti-gram-positive bacterial) appear in approximately 40% of cases. To evaluate the model's performance, the dataset was split into training, validation, and test sets (60%, 10%, and 30% respectively). This split allowed for experimentation to determine the optimal number of examples retrieved from the database, the most effective embedder, and the benefits of multi-modal representation. The MMC metric was used to compare the performance of different experimental configurations.

The results of the experiments provide several key insights. Bar charts 702 and 704 suggest that a relatively low number of retrieved examples (e.g., set at 5 for subsequent experiments) and the 150M parameter version of ESM2 embedder may be effective. Bar chart 706 demonstrates that among the different modalities, the embedding (method RAC-E) may be impactful, with a slight improvement when adding physicochemical features (method RAC-E-PCF). The addition of contact map information (method RAC-E-PCF-CM) appears to have a minor impact when combined with the other two modalities. Bar chart 708 highlights a significant performance improvement over previous baselines, which may be attributed to the RAC strategy, the use of pre-trained Protein Language Models (PLMs), and the incorporation of physicochemical features. The reported results reflect performance on the test dataset after hyperparameter optimization using the train/validation split.

Table 2 below provides a detailed comparison of the multimodal variants of the RAC approach with the TiAmp baselines. Table 2 presents performance metrics including Sensitivity, Specificity, Balanced Accuracy (BA), Geometric Mean (GMean), and MCC for different methods.

TABLE 2
Performance of the Multi-Modal Varients

| Method | Sensitivity | Specificity | BA | GMean | MCC |
|---|---|---|---|---|---|
| RF | 0.498 | 0.953 | 0.7255 | 0.688 | 0.3763 |
| TiAmp | 0.568 | 0.92 | 0.744 | 0.722 | 0.5256 |
| TiAmp + ASL | 0.739 | 0.857 | 0.798 | 0.795 | 0.5663 |
| RAC - E | 0.835 | 0.94 | 0.836 | 0.887 | 0.674 |
| RAC - E - PCF | 0.805 | 0.935 | 0.855 | 0.870 | 0.691 |
| RAC - E - | 0.766 | 0.956 | 0.841 | 0.861 | 0.692 |
| PCF - CM | | | | | |

The RAC variants (RAC-E, RAC-E-PCF, RAC-E-PCF-CM) show improved performance across most metrics compared to the baseline methods (RF, TiAmp, TiAmp+ASL). This comprehensive comparison may allow for a more nuanced understanding of how each variant performs across different evaluation criteria.

These visualizations and data demonstrate the effectiveness of the proposed multimodal RAC approach for protein function prediction, particularly in the context of antimicrobial peptide function prediction. The results indicate that the new method may outperform previous approaches, with the combination of embedding (E) and physicochemical features (PCF) providing significant improvements. The use of multiple modalities in the RAC approach may allow for a more comprehensive representation of protein characteristics, potentially leading to more accurate predictions. The improved performance across various metrics suggests that this method may be valuable for researchers and practitioners working in the field of protein function prediction and antimicrobial peptide discovery.

In example embodiments, a method and system to generate proteins or their parts or fragments are provided. The embedding model 108 trained on protein sequences or amino acid sequences as detailed above is used to generate new proteins. In example embodiments, the embedding model 108 is trained on a large dataset of public protein sequences with a masked language learning model. The Masked Language Model includes hiding/masking parts of known protein sequences and getting the model to predict the missing parts. The model therefore creates or generates a new sequence and also augments each sequence with a corresponding vector of physiochemical features and/or a corresponding contact map matrix representing the 3D structure of protein. In example embodiments, physiochemical features and contact maps are combined by early fusion i.e., by concatenating the physiochemical features and contact maps to the protein sequence in a same embedding space. The physiochemical features may contain any combination of the following: BLOSUM Indices, Cruciani Properties, FASGAI vectors, Kidera factors, MS-WHIM scores, PCP properties, ProtFP descriptors, Sneath vectors, ST-scales, T-scales, VHSE-scales, Z-scales, Aliphatic Index, Autocorrelation, Autocovariance, Boman Index, Lehninger Charge, Hydrophobic Moment α, Hydrophobic Moment β, Hydrophobicity, Instability Index, Isoelectric Point, Mass Shift, Molecular Weight, Mass over charge ratio.

After the embedded model 108 is trained, it may be used in two different modes. One of the modes is “Generation Mode”. In Generation Mode, either an existing protein is selected to generate its variants or missing parts, if any, or a protein is generated based on desired functionalities and properties without using any existing protein.

Yet, another mode is “Embedding Mode” in which the embedding model 108 outputs a protein representation that may be used as basis to train other models for further tasks such as protein structure prediction, functional annotation, solubility prediction, fluorescence intensive prediction, antigen, antigen binding site prediction, epitope prediction, protein-protein interaction prediction, contact map prediction, physiochemical feature prediction and toxicity prediction.

While the foregoing is directed to example embodiments described herein, other and further example embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the example embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed example embodiments, are example embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method of predicting protein functions and interactions, comprising:
identifying, by a computing system, a first protein and a second protein for analysis;
generating, by the computing system, a first embedded representation of a first amino acid sequence of the first protein and a second embedded representation of a second amino acid sequence of the second protein;
generating, by the computing system, a first contact map prediction for the first protein based on the first amino acid sequence and a second contact map prediction for the second protein based on the second amino acid sequence;
generating, by the computing system, first physiochemical features associated with the first protein, and second physiochemical features associated with the second protein; and
generating, by the computing system, a prediction score for a protein function or interaction between the first protein and the second protein based on the first embedded representation, the second embedded representation, the first contact map prediction, the second contact map prediction, the first physiochemical features and the second physiochemical features.

2. The method of claim 1, further comprising:
generating, by the computing system, the first contact map prediction for the first protein based on the first amino acid sequence and the second contact map prediction for the second protein based on the second amino acid sequence by:
estimating first three-dimensional distances between first amino acid residues in the first amino acid sequence; and
estimating second three-dimensional distances between second amino acid residues in the second amino acid sequence.

3. The method of claim 1, further comprising:
generating, by the computing system, the first physiochemical features associated with the first protein, and the second physiochemical features associated with the second protein by passing the first amino acid sequence and the second amino acid sequence through a set of descriptors.

4. The method of claim 1, further comprising:
generating, by the computing system, the prediction score for the protein function or interaction between the first protein and the second protein by:
generating an embedded first protein amino acid sequence by encoding the first embedded representation of the first amino acid sequence, the first contact map prediction, and the first physiochemical features; and
generating an embedded second protein amino acid sequence by encoding the second embedded representation of the second amino acid sequence, the second contact map prediction, and the second physiochemical features.

5. The method of claim 4, further comprising:
concatenating the embedded first protein amino acid sequence and the embedded second protein amino acid sequence.

6. The method of claim 5, further comprising:
generating, via a feed forward neural network, the prediction score based on the concatenated embedded first protein amino acid sequence and the embedded second protein amino acid sequence.

7. The method of claim 1, further comprising:
receiving, by the computing system, input data comprising a protein sequence, a number of neighbors, and a classification threshold; and
initializing, by the computing system, a set of retrieved neighbors;
for each modality of a plurality of modalities by:
extracting, by the computing system, a modality representation of the protein sequence,
retrieving, by the computing system, neighbors using the modality representation and a corresponding vector database, and
adding, by the computing system, the retrieved neighbors to the set of retrieved neighbors;
computing, by the computing system, a probability for each label index based on the set of retrieved neighbors; and
outputting, by the computing system, predicted labels having probabilities greater than or equal to the classification threshold.

8. The method of claim 1, wherein the first protein is a T-cell receptor (TCR) and the second protein is an epitope.

9. The method of claim 8, wherein the prediction score represents a binding affinity between the TCR and the epitope.

10. The method of claim 8, wherein the first physiochemical features associated with the TCR and the second physiochemical features associated with the epitope include one or more of: hydrophobicity, charge, size, and polarity.

11. The method of claim 8, wherein generating the first contact map prediction for the TCR and the second contact map prediction for the epitope comprises estimating spatial relationships between amino acid residues in the TCR and the epitope, respectively.

12. A non-transitory computer-readable medium storing instructions that, when executed by one or more processors, cause the one or more processors to perform operations for predicting protein functions and interactions, the operations comprising:
identifying a first protein and a second protein for analysis;
generating a first embedded representation of a first amino acid sequence of the first protein and a second embedded representation of a second amino acid sequence of the second protein;
generating a first contact map prediction for the first protein based on the first amino acid sequence and a second contact map prediction for the second protein based on the second amino acid sequence;
generating first physiochemical features associated with the first protein, and second physiochemical features associated with the second protein; and
generating a prediction score for a protein function or interaction between the first protein and the second protein based on the first embedded representation, the second embedded representation, the first contact map prediction, the second contact map prediction, the first physiochemical features and the second physiochemical features.

13. The non-transitory computer-readable medium of claim 12, wherein the operations further comprise:
generating the first contact map prediction for the first protein based on the first amino acid sequence and the second contact map prediction for the second protein based on the second amino acid sequence by:
estimating first three-dimensional distances between first amino acid residues in the first amino acid sequence; and
estimating second three-dimensional distances between second amino acid residues in the second amino acid sequence.

14. The non-transitory computer-readable medium of claim 12, wherein the operations further comprise:
generating the first physiochemical features associated with the first protein, and the second physiochemical features associated with the second protein by passing the first amino acid sequence and the second amino acid sequence through a set of descriptors.

15. The non-transitory computer-readable medium of claim 12, wherein the operations further comprise:
generating the prediction score for the protein function or interaction between the first protein and the second protein by:
generating an embedded first protein amino acid sequence by encoding the first embedded representation of the first amino acid sequence, the first contact map prediction, and the first physiochemical features; and
generating an embedded second protein amino acid sequence by encoding the second embedded representation of the second amino acid sequence, the second contact map prediction, and the second physiochemical features.

16. The non-transitory computer-readable medium of claim 15, wherein the operations further comprise:
concatenating the embedded first protein amino acid sequence and the embedded second protein amino acid sequence.

17. The non-transitory computer-readable medium of claim 16, wherein the operations further comprise:
generating, via a feed forward neural network, the prediction score based on the concatenated embedded first protein amino acid sequence and the embedded second protein amino acid sequence.

18. The non-transitory computer-readable medium of claim 12, wherein the operations further comprise:
receiving input data comprising a protein sequence, a number of neighbors, and a classification threshold;
initializing a set of retrieved neighbors; and
for each modality of a plurality of modalities:
extracting a modality representation of the protein sequence,
retrieving neighbors using the modality representation and a corresponding vector database, and
adding the retrieved neighbors to the set of retrieved neighbors;
computing a probability for each label index based on the set of retrieved neighbors; and
outputting predicted labels having probabilities greater than or equal to the classification threshold.

19. The non-transitory computer-readable medium of claim 12, wherein the first protein is a T-cell receptor (TCR) and the second protein is an epitope.

20. The non-transitory computer-readable medium of claim 19, wherein the prediction score represents a binding affinity between the TCR and the epitope.

21. The non-transitory computer-readable medium of claim 19, wherein the first physiochemical features associated with the TCR and the second physiochemical features associated with the epitope include one or more of: hydrophobicity, charge, size, and polarity.

22. The non-transitory computer-readable medium of claim 19, wherein generating the first contact map prediction for the TCR and the second contact map prediction for the epitope comprises estimating spatial relationships between amino acid residues in the TCR and the epitope, respectively.

23. A system for predicting protein functions and interactions, comprising:
a processor; and
a memory having programming instructions stored thereon, which, when executed by the processor, perform operations comprising:
identifying a first protein and a second protein for analysis;
generating a first embedded representation of a first amino acid sequence of the first protein and a second embedded representation of a second amino acid sequence of the second protein;
generating a first contact map prediction for the first protein based on the first amino acid sequence and a second contact map prediction for the second protein based on the second amino acid sequence;
generating first physiochemical features associated with the first protein, and second physiochemical features associated with the second protein; and
generating a prediction score for a protein function or interaction between the first protein and the second protein based on the first embedded representation, the second embedded representation, the first contact map prediction, the second contact map prediction, the first physiochemical features and the second physiochemical features.

24. The system of claim 23, wherein the operations further comprise:
generating the first contact map prediction for the first protein based on the first amino acid sequence and the second contact map prediction for the second protein based on the second amino acid sequence by:
estimating first three-dimensional distances between first amino acid residues in the first amino acid sequence; and
estimating second three-dimensional distances between second amino acid residues in the second amino acid sequence.

25. The system of claim 23, wherein the operations further comprise:
generating the first physiochemical features associated with the first protein, and the second physiochemical features associated with the second protein by passing the first amino acid sequence and the second amino acid sequence through a set of descriptors.

26. The system of claim 23, wherein the operations further comprise:
generating the prediction score for the protein function or interaction between the first protein and the second protein by:
generating an embedded first protein amino acid sequence by encoding the first embedded representation of the first amino acid sequence, the first contact map prediction, and the first physiochemical features; and
generating an embedded second protein amino acid sequence by encoding the second embedded representation of the second amino acid sequence, the second contact map prediction, and the second physiochemical features.

27. The system of claim 23, wherein the operations further comprise:
concatenating the embedded first protein amino acid sequence and the embedded second protein amino acid sequence; and
generating, via a feed forward neural network, the prediction score based on the concatenated embedded first protein amino acid sequence and the embedded second protein amino acid sequence.

28. The system of claim 23, wherein the operations further comprise:
receiving input data comprising a protein sequence, a number of neighbors, and a classification threshold;
initializing a set of retrieved neighbors; and
for each modality of a plurality of modalities:
extracting a modality representation of the protein sequence,
retrieving neighbors using the modality representation and a corresponding vector database, and
adding the retrieved neighbors to the set of retrieved neighbors;
computing a probability for each label index based on the set of retrieved neighbors; and
outputting predicted labels having probabilities greater than or equal to the classification threshold.

29. The system of claim 23, wherein the first protein is a T-cell receptor (TCR) and the second protein is an epitope.

30. The system of claim 29, wherein the prediction score represents a binding affinity between the TCR and the epitope.

31. The system of claim 30, wherein the first physiochemical features associated with the TCR and the second physiochemical features associated with the epitope include one or more of:
hydrophobicity, charge, size, and polarity.

32. The system of claim 31, wherein generating the first contact map prediction for the TCR and the second contact map prediction for the epitope comprises estimating spatial relationships between amino acid residues in the TCR and the epitope, respectively.
